(19) United States
(12) Patent Application Publication
(10) Pub. No.: US 2008/0242703 A1
(43) Pub. Date: Oct. 2, 2008

(54) Pharmaceutical compositions for the treatment of hearing loss

(75) Inventors: Chuan-Jen Hsu, Taipei (TW); Pei-Jer Chen, Taipei (TW); Chen-Chi Wu, Taipei (TW)

Correspondence Address: BACON & THOMAS, PLLC, 625 SLATERS LANE, FOURTH FLOOR, ALEXANDRIA, VA, 22314-1176, US

(73) Assignee: National Taiwan University, Taipei (TW)

(21) Appl. No.: 11/898,829

(22) Filed: Sep. 17, 2007

(30) Foreign Application Priority Data
Apr. 2, 2007 (TW) 096111541

(51) Int. Cl. A61K 31/4439; A61K 31/4184; A61P 27/16

(52) U.S. Cl. 514/338; 514/394

(57) ABSTRACT

The present invention provides a pharmaceutical composition for the treatment of hearing loss. The pharmaceutical composition of the present invention comprises a proton pump inhibitor (PPI), and is useful to decreasing acute or chronic hearing loss of patients related to the mutation or degenerative dysfunction of SLC26A4 gene.

### BACKGROUND OF THE INVENTION

1. Field of the Invention

The present invention relates to a pharmaceutical composition, and more particularly to a pharmaceutical composition for the treatment of hearing loss.

2. Description of the Prior Art

The hearing of human being is the process, function, or power of perceiving sound. Because of genetic diseases, fevers, pathological changes of acoustic nerves, traumas, or other innate or acquired deficiency of hearing system, the ability of hearing may be damaged, namely deafness or hearing loss. Otherwise, the term deafness or hearing loss may also indicate that one can hear sound, but cannot identify or understand the meaning of that sound.

According to the properties, there are two types of hearing loss, reversible or irreversible. In the case of reversible hearing loss, it is generally cured through the treatment of eardrum or ear infections. Some causes inflammation of ear, e.g. otitis externa or tympanitis, and some are due to the growth of foreign matters, breaking of ossicles, or otosclerosis. Most symptoms can be cured by medicines or surgeries to restore hearing.

Furthermore, the irreversible hearing loss are divided into two types, one is naive irreversible hearing loss, also called pre-lingual hearing loss, which happens before birth, and impedes baby's ability to learn language. Another is acquired irreversible hearing loss, also called post-lingual hearing loss.

There are several reasons associated with naive irreversible hearing loss, for example, genetic factors, bacterial or viral infections, parturient factors. Heredity is a cause of hearing loss, but not all inherited forms of hearing loss take place at birth. For example, hearing loss caused by dominant inheritance results in one ear or two ear deaf, and the patient will lose his hearing when he is getting older. As for recessive inheritance or sex-linkage inheritance, hearing loss is happened in both ears, and results in severe or extra-severe disability. Naïve bacterial or viral infections usually affect the fetus by bacteria and virus in the female parent. For example, Rubella virus, cytomegalovirus (CMV), Treponema pallidum can affect the fetus and result in hearing loss. Parturient factors include asphyxia, pathologic jaundice, and head damage of neonatal babies during parturition.

Moreover, reasons related to acquired irreversible hearing loss are described as follows, for example, fracture of temporal bone, which may cause conductive hearing loss (reversible), or sensorineural hearing loss (irreversible); diseases such as acute upper respiratory infection (pneumonia), herpes, cephalitis, measles; toxic drugs such as aminoglycosides, quinine, salicylate, hydragogue. And it is also known that one repeated exposures to extremely loud noise for a long time can lead to permanent, incurable hearing loss.

With the advances in molecular genetics, the nature of hereditary hearing loss has started to be unraveled. A plurality of deafness genes are discovered in the past years, for example, Cx26 (GJB2), Cx31 (GJB3), Cx30 (GJB6), Cx32 (GJB1), DIAPH1, MYO7A, MYO15, OTOF, SLC26A4 (PDS), etc. Among these genes, certain genetic mutations were noted to be extraordinarily popular in the hearing-impaired population, e.g. SLC26A4 gene is one related to hereditary hearing loss.

In 1997, SLC26A4 (PDS) gene is identified as the disease gene of Pendred syndrome, a condition characterized by hearing loss and an enlarged thyroid gland (goiter). In addition, patients with Pendred syndrome usually have an enlarged vestibular aqueduct or Mondini's dysplasia, a malformed cochlea. Besides, patients with SLC26A4 gene mutation also have acute fluctuating hearing loss.

There exists some conventional methods associated with treating hearing loss caused by SLC26A4 (PDS) gene mutation, for example, steroid or intracranial-pressure-lowering-medication. However, these methods usually can not achieve satisfactory and predictable outcomes.

In order to overcome the foresaid drawbacks in the prior arts, the present invention provides a pharmaceutical composition for the treatment of hearing loss.

### SUMMARY OF THE INVENTION

It is an aspect of the present invention to provide a pharmaceutical composition for the treatment of hearing loss, particularly for the treatment of acute hearing loss.

According to a preferred embodiment, it is another aspect of the present invention to provide a pharmaceutical composition for the treatment of hearing loss. The pharmaceutical composition includes a proton pump inhibitor (PPI), and is useful for the treatment of hearing loss caused by the malfunction of SLC26A4 gene.

It is another aspect of the present invention to apply well-known proton pump inhibitor drugs to a new disease. Proton pump inhibitor drugs are used to be applied to clinical indications, e.g. peptic ulcers, refluxesophagitis, Helicobacter pylori, and MALToma. The new usage of proton pump inhibitor drugs is applied to treat acute or chronic hearing loss of a patient caused by mutation or malfunction of SLC26A4 gene.

It is another aspect of the present invention to provide a method of preparing a pharmaceutical composition for the treatment of hearing loss, including the following steps: preparing main material of the pharmaceutical composition, including a proton pump inhibitor according to the present invention; and forming a coating layer on the surface of the main material to compose the pharmaceutical composition.

In a preferred embodiment, the pharmaceutical composition of the present invention further includes a pharmaceutically acceptable carrier and an excipient.

In a preferred embodiment, the proton pump inhibitor according to the present invention is a derivative of benzimidazole.

Preferably, proton pump inhibitor is selected from the group consisting of omeprazole, lansoprazole, pantoprazole, rabeprazole, esomerprazole, pariprazole, and leminoprazole, and alkaline salts, enantiomers, alkaline salts of said enantiomers, isomers, alkaline salts of said isomers, or the mixtures thereof.

More preferably, the proton pump inhibitor is omeprazole, lansoprazole, pantoprazole, rabeprazole, or the mixtures thereof.

It is another aspect of the present invention to provide a method for the treatment of hearing loss caused by the malfunction of SLC26A4 gene. The method according to the present invention includes administering to a patient suffering from hearing loss an effective amount of the pharmaceutical compositions according to the present invention.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1a is a schematic view illustrating the structure of one proton pump inhibitor, omeprazole in the present invention;

FIG. 1b is a schematic view illustrating the structure of another proton pump inhibitor, lansoprazole in the present invention;

FIG. 1c is a schematic view illustrating the structure of another proton pump inhibitor, pantoprazole in the present invention;

FIG. 1d is a schematic view illustrating the structure of another proton pump inhibitor, rabeprazole in the present invention; and

FIG. 2 is a schematic view illustrating the flowchart of the method of preparing the pharmaceutical composition according to the present invention.

### DESCRIPTION OF THE PREFERRED EMBODIMENT

The following is a description of the present invention and the invention will firstly be described with reference to one exemplary structure. Some variations will then be described as well as advantages of the present invention. A preferred method of fabrication will then be discussed; also, an alternate, asymmetric embodiment will then be described along with the variations in the process flow to fabricate this embodiment.

In a preferred embodiment, the present invention provides a pharmaceutical composition for the treatment of hearing loss, and the pharmaceutical composition includes a proton pump inhibitor.

The term “hearing loss” or “deafness” described herein includes but not limit to, hearing loss or deficient hearing caused by innate or acquired factors, especially hearing loss resulted from genetic reasons. Further, the term “hearing loss” described herein does not mean a specific level of hearing loss.

Furthermore, the term “abnormal gene” described herein indicates several kinds of gene mutations, including but not limit to, deletion, insertion, or point mutation. The term “abnormal gene” described herein further represents a gene which can transcript and translate a protein with structural or functional abnormalities. In other words, SLC26A4 gene, which transcript and translate a pendrin protein with structural or functional abnormalities, is included in the field of “hearing loss” described in the present invention.

In some preferred embodiments, a proton pump inhibitor can be derivatives of benzimidazole, which is described in the U.S. Pat. Nos. 4,045,563, 4,255,431, 4,628,098, 4,686,230, 4,758,579, 4,965,269, 5,021,433, 5,430,042, 5,708,017 and 6,093,734.

In certain embodiments of the present invention, the proton pump inhibitor is selected from the group consisting of omeprazole, lansoprazole, pantoprazole, rabeprazole, esomerprazole, pariprazole, and leminoprazole, and alkaline salts, enantiomers, alkaline salts of said enantiomers, isomers, alkaline salts of said isomers, or the mixtures thereof. The proton pump inhibitor can be a naive form or its pharmaceutically acceptable salt.

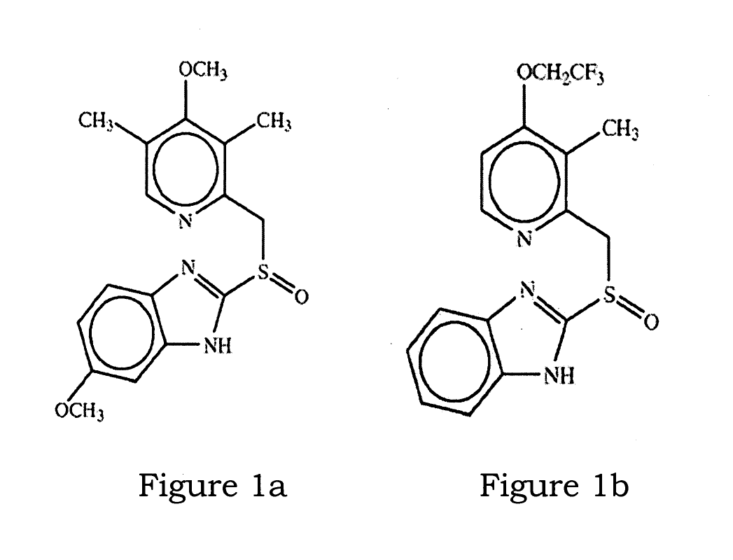

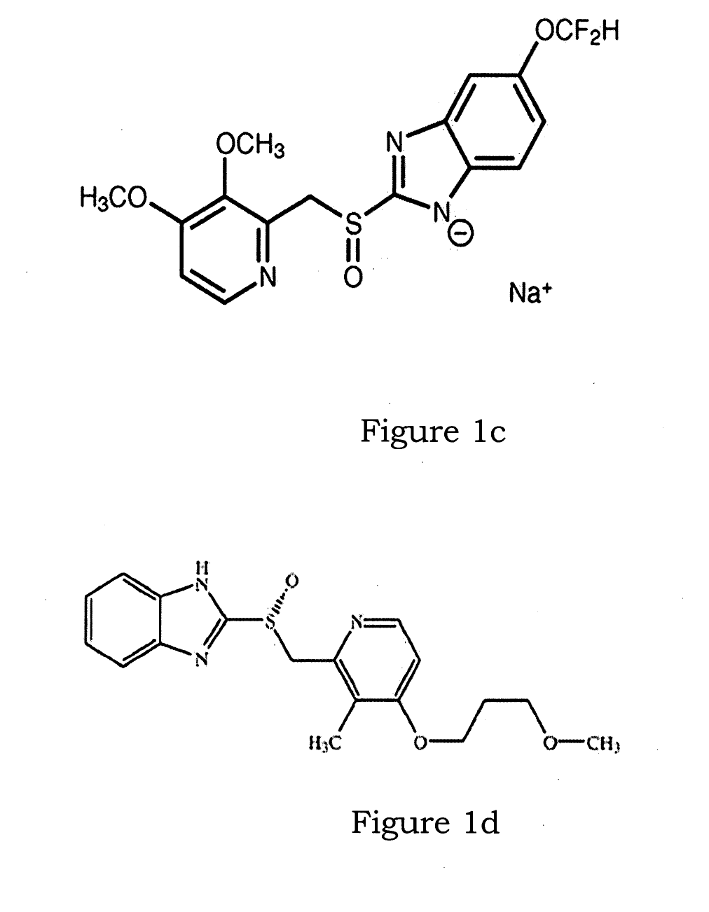

Referring to FIG. 1a to 1d will illustrate the structures of proton pump inhibitors according to the present invention.

As shown in FIG. 1a, in certain embodiments the proton pump inhibitor can be omeprazole, alkaline salts, enantiomers, alkaline salts of said enantiomers, isomers, alkaline salts of said isomers, or the mixtures thereof, which is described in the U.S. Pat. No. 4,255,431 and U.S. Pat. No. 5,693,818. For example, the proton pump inhibitor can be the magnesium salt of omeprazole.

As shown in FIG. 1b, in the certain embodiments the proton pump inhibitor can be lansoprazole, alkaline salts, enantiomers, alkaline salts of said enantiomers, isomers, alkaline salts of said isomers, or the mixtures thereof, which is described in the U.S. Pat. No. 4,628,098.

As shown in FIG. 1c, in the certain embodiments the proton pump inhibitor can be pantoprazole, alkaline salts, enantiomers, alkaline salts of said enantiomers, isomers, alkaline salts of said isomers, or the mixtures thereof, which is described in the U.S. Pat. No. 4,758,579.

As shown in FIG. 1d, in the certain embodiments the proton pump inhibitor can be rabeprazole, alkaline salts, enantiomers, alkaline salts of said enantiomers, isomers, alkaline salts of said isomers, or the mixtures thereof, which is described in the U.S. Pat. No. 5,045,552.

In addition, the foresaid benzimidazole derivatives, the proton pump inhibitor used in the pharmaceutical compositions according to the present invention can be other suitable pharmaceutical acceptable materials.

The pharmaceutical composition of the present invention is formulated to be compatible with its intended route of administration. For example, the composition can be administered by an enteral mode, e.g., orally administration or infusion. The pharmaceutical composition of the present invention can be formulated in the form of tablets, coated tablets, sugar-coated tablets, capsules, powders, pills, syrups, solutions, emulsions or suspensions.

In another preferred embodiment, for example, the composition of the present invention can be administered by a parenteral mode, e.g., intravenous, subcutaneous, intraperitoneal, or intramuscular injection.

The pharmaceutical compositions according to the present invention can further include a carrier, especially a pharmaceutically acceptable carrier. Suitable pharmaceutically acceptable carriers used in the present invention include, but not limited to water, sterilized aqua solution, salt solution, e.g., NaCl, saline, buffered saline, alcohol, glycerol, ethanol, water soluble arabic gum, vegetable oil, benzyl alcohol, polyethylene glycol, glue, carbohydrate, such as lactose, amylose, dextrose, magnesium stearate, talc, silicic acid, paraffin, essence oil, stearate, hydroxymethyl cellulose, polyvinylpyrrolidone, and the mixtures thereof.

The pharmaceutical compositions according to the present invention can further include pharmaceutically inert, inorganic or organic excipients. For oral administration, tablets, coated tablets, sugar-coated tablets, capsules, powders, pills, or similitude can include one or more following excipients: a diluting agent or a filler, a binder, a disintegrating agent, a lubricant, a glidant, a taste masking agent or a flavoring agent.

For example, a diluting agent or a filler includes starches such as starch, potato starch, corn starch, and cellulose, crystalline cellulose, lactose, gelatin, calcium hydrogenphosphate, and polyvinyl pyrrolidone or macrogol.

A binder includes, for example, the same compounds as the above diluting agents.

A disintegrating agent includes, for example, the same compounds as in the foresaid excipients, and derivative of starches and celluloses such as cross carmellose sodium, sodium carboxymethylstarch, and crosslinked polyvinyl pyrrolidone.

A lubricant, also called a coating agent, includes, for example, talc, magnesium stearate, ethylcellulose, hydroxypropylcellulose, hydroxypropyl-methylcellulose, shellac, carnauba wax, and paraffin.

A glidant includes, for example, silica gel.

A taste masking agent or a flavoring agent includes sweeteners, acidifiers and flavors usually used, for example, sucrose or saccharin, mint or cherry flavors.

Besides, a suitable excipient for preparing soft capsules includes, for example, vegetable oil, wax, fat, semi-solid or liquid polyol. A suitable excipient for preparing solutions or syrups, includes, for example, water, polyol, sucrose, invert sugar, dextran, and glucose.

For parenteral administration, the pharmaceutical composition of the present invention can includes aqueous excipient and non-aqueous excipient. The aqueous excipient includes ethanol, water, buffer medium and analogues, and mixtures thereof. The non-aqueous excipient includes ethanol and ethylene glycol, e.g. ethanol and polyethylene glycol; oil, e.g. vegetable oil; fatty acid, fatty acid ester and analogues thereof. Some other excipient can be added, for example, detergent, e.g. hydroxypropyl cellulose; isotonic agent, e.g. sodium chloride; fluid and nutrient supplement; electrolyte supplement; controlled releasing agent with active materials, e.g. aluminum monostearate and copolymer; antibacterial, e.g. chlorobutanol or phenol; buffer solution and analogues.

Further, the pharmaceutical composition of the present invention can includes aseptics, lytic agents, adhesives, stabilizers, humectants, emulsifying agents, sweeteners, coloring agents, flavors, salts for changing osmosis, buffer solutions, film-coating, or antioxidant, other therapeutic matters, if needed.

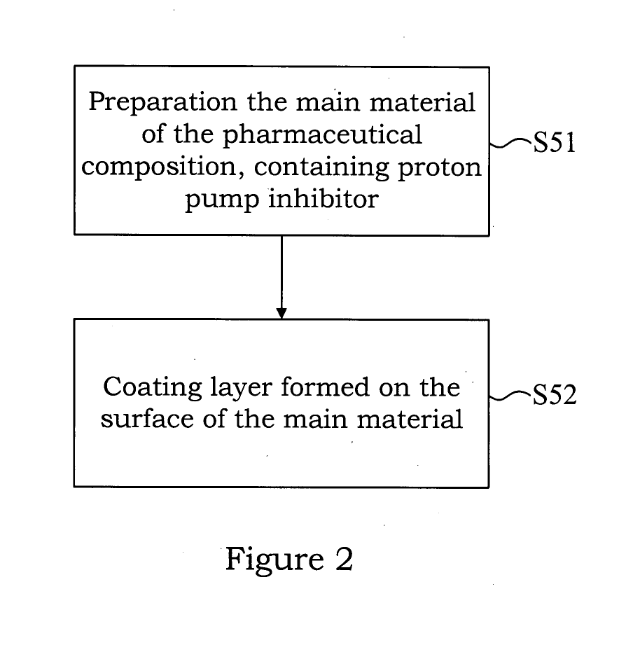

Referring to FIG. 2, it is a schematic view illustrating the flowchart of the method of preparing the pharmaceutical composition according to a preferred embodiment of the present invention.

As shown in the step S51 in FIG. 2, the method of preparing the pharmaceutical composition according to the present invention includes the following steps. Firstly, preparation main material of the pharmaceutical composition will contain a proton pump inhibitor.

According to the step S52 shown in FIG. 2, a coating layer is formed on the surface of foresaid main material to produce the pharmaceutical composition of the present invention.

There are some embodiments to illustrate the effect of the pharmaceutical composition of the present invention treating hearing loss related to abnormal SLC26A4 gene.

The first Preferred Embodiment: administrating a proton pump inhibitor drug to a patient with the mutation of SLC26A4 gene

In this embodiment, three hearing loss patients are identified that they have SLC26A4 gene mutation. The pharmaceutical compositions of the present invention are administered to those patients.

The three patients are administered eight times while acute hearing loss happened in this embodiment. During the eight treatments, hearing loss of patients are notably ameliorated. The result is shown in Table 1.

The second Preferred Embodiment: administrating steroids to a patient with the mutation of SLC26A4 gene

In this embodiment, there are also three hearing loss patients identified that they have SLC26A4 gene mutation. Conventional steroid drugs are administered to those patients. The three patients are administered eight times while acute hearing loss happened in this embodiment. During the eight treatments, hearing loss of patients are not improved.

The described embodiments illustrate that the pharmaceutical composition of the present invention can effectively improve the hearing loss of patients, especially for the acute hearing loss caused by the malfunction of SLC26A4 gene.

The SLC26A4 gene, also called PDS gene, produces a protein called pendrin, which is a transporter protein of chloride and iodine ions. It is said that the pendrin regulates ion concentration and endolymph in the inner ear. In the past researches of pendrin, the function of regulating bicarbonate, HCO₃—, was ignored. In the cell of inner ear, the transportation of bicarbonate is a antagonism toward the proton pump of inner ear epitheliums. Maintaining the correct level of these ions is important for supporting reactions that are critical to the hearing process and for determining the amount of fluid that bathes the inner ear. The fluid level appears to be particularly important during development of the inner ear, as it may influence the shape of the bony structures. The mutation of SLC26A4 gene will result in inner ear malformation and deafness. Therefore, SLC26A4 mutations likely impair pendrin activity, which upsets the balance of ions between endolymph and tissue of the inner ear. Meanwhile, steps in the hearing process are disrupted, causing hearing loss, and abnormal fluid levels affect inner ear structures.

According to the present invention, a proton pump inhibitor can be applied to improve acute or chronic hearing loss of patients related to the mutation or malfunction of SLC26A4 gene. Consequently, one object of the present invention is provide proton pump inhibitor drugs to improve acute or chronic hearing loss of patients related to the mutation or malfunction of SLC26A4 gene.

The patients related to the mutation of SLC26A4 gene suffer from fluctuating hearing loss, which may result from disequilibrium between acid and base in the inner ear. For acid-base homeostasis in the inner ear, proton pumps have antagonistic effects to pendrin. If a proton pump inhibitor is applied to patients related to the mutation of SLC26A4 gene to block proton pumps, both pendrin and proton pumps are not well-function, and the acid-base homeostasis in the inner ear of patients is become stable. Therefore, acquired or degenerative deafness related the mutation of SLC26A4 gene can also be ameliorated through proton pump inhibitors, i.e. the pharmaceutical composition of the present invention.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

It is understood that various other modifications will be apparent to and can be readily made by those skilled in the art without departing from the scope and spirit of this invention. Accordingly, it is not intended that the scope of the claims appended hereto be limited to the description as set forth herein, but rather that the claims be construed as encompassing all the features of patentable novelty that reside in the present invention, including all features that would be treated as equivalents thereof by those skilled in the art to which this invention pertains.

What is claimed is:

1. A pharmaceutical composition for the treatment of hearing loss comprises a proton pump inhibitor (PPI).

2. The pharmaceutical composition according to claim 1, further comprising a pharmaceutically acceptable carrier and an excipient.

3. The pharmaceutical composition according to claim 1, wherein said proton pump inhibitor is a derivative of benzimidazole.

4. The pharmaceutical composition according to claim 3, wherein said proton pump inhibitor is selected from the group consisting of omeprazole, lansoprazole, pantoprazole, rabeprazole, esomerprazole, pariprazole, and leminoprazole, and alkaline salts, enantiomers, alkaline salts of said enantiomers, isomers, alkaline salts of said isomers, or the mixtures thereof.

5. The pharmaceutical composition according to claim 4, wherein said proton pump inhibitor is an effective amount of omeprazole, alkaline salts, enantiomers, alkaline salts of said enantiomers, isomers, alkaline salts of said isomers, or the mixtures thereof.

6. The pharmaceutical composition according to claim 4, wherein said proton pump inhibitor is an effective amount of lansoprazole, alkaline salts, enantiomers, alkaline salts of said enantiomers, isomers, alkaline salts of said isomers, or the mixtures thereof.

7. The pharmaceutical composition according to claim 4, wherein said proton pump inhibitor is an effective amount of pantoprazole, alkaline salts, enantiomers, alkaline salts of said enantiomers, isomers, alkaline salts of said isomers, or the mixtures thereof.

8. The pharmaceutical composition according to claim 4, wherein said proton pump inhibitor is an effective amount of rabeprazole, alkaline salts, enantiomers, alkaline salts of said enantiomers, isomers, alkaline salts of said isomers, or the mixtures thereof.

9. The pharmaceutical composition according to claim 1, wherein said hearing loss is related to the malfunction of SLC26A4 gene.

10. The pharmaceutical composition according to claim 1, wherein said hearing loss is related to an abnormal pendrin.

11. A pharmaceutical composition for the treatment of hearing loss comprises a proton pump inhibitor (PPI), wherein said hearing loss is related to abnormal SLC26A4 gene.

12. The pharmaceutical composition according to claim 11, further comprising a pharmaceutically acceptable carrier and an excipient.

13. The pharmaceutical composition according to claim 11, wherein said proton pump inhibitor is a derivative of benzimidazole.

14. The pharmaceutical composition according to claim 13, wherein said proton pump inhibitor is selected from the group consisting of omeprazole, lansoprazole, pantoprazole, rabeprazole, esomerprazole, pariprazole, and leminoprazole, and alkaline salts, enantiomers, alkaline salts of said enantiomers, isomers, alkaline salts of said isomers, or the mixtures thereof.

15. The pharmaceutical composition according to claim 14, wherein said proton pump inhibitor is omeprazole, lansoprazole, pantoprazole, or rabeprazole.

16. A method of preparing a pharmaceutical composition for the treatment of hearing loss caused by abnormal SLC26A4 gene, comprising:
preparing main material of said pharmaceutical composition, including a proton pump inhibitor; and
forming a coating layer on the surface of said main material to produce said pharmaceutical composition.

17. The method according to claim 16, wherein said proton pump inhibitor is omeprazole, lansoprazole, pantoprazole, or rabeprazole.

18. The method according to claim 16, wherein said coating layer comprises a pharmaceutically acceptable carrier and an excipient.

19. A method for the treatment of hearing loss caused by abnormal SLC26A4 gene, comprising administering to a patient suffering from said hearing loss an effective amount of the pharmaceutical compositions of claim 1 and claim 11.
